(19) United States
(12) Patent Application Publication
(10) Pub. No.: US 2009/0005586 A1
(43) Pub. Date: Jan. 1, 2009

(54) MIXED METAL OXIDE CATALYSTS FOR THE AMMOXIDATION OF PROPANE AND ISOBUTANE

(76) Inventors: James F. Brazdil, JR., Glen Ellyn, IL (US); Bruce I. Rosen, Park Ridge, IL (US); Eric Moore, Wheaton, IL (US); Joseph P. Bartek, Wheaton, IL (US); Claus Lugmair, San Jose, CA (US); Benjamin Mork, New Port Beach, CA (US)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 11/771,278

(22) Filed: Jun. 29, 2007

(51) Int. Cl. C07C 253/24; B01J 21/08; B01J 23/10; B01J 27/057

(52) U.S. Cl. 558/319; 423/263; 502/215; 502/242; 502/247; 502/302

(57) ABSTRACT

A catalyst composition comprising molybdenum, vanadium, and antimony, and at least one other element selected from the group consisting of praseodymium, neodymium, samarium, europium, gadolinium, terbium, dysprosium, holmium, erbium, thulium, ytterbium, and lutetium. Such catalyst compositions are effective for the gas-phase conversion of propane to acrylonitrile and isobutane to methacrylonitrile (via ammoxidation).

### BACKGROUND OF THE INVENTION

1. Field of the Invention

The present invention generally relates to catalyst compositions, methods of preparing such catalyst compositions, and methods of using such catalyst compositions for the gas-phase conversion of propane to acrylonitrile and isobutane to methacrylonitrile (via ammoxidation) or of propane to acrylic acid and isobutane to methacrylic acid (via oxidation).

The invention particularly relates to catalyst compositions, methods of preparing such catalyst compositions, and methods of using such catalyst compositions, where in each case, the same comprises molybdenum, vanadium, and antimony.

2. Description of the Prior Art

Generally, the field of the invention relates to catalysts containing molybdenum, vanadium, and antimony that have been shown to be effective for conversion of propane to acrylonitrile and isobutane to methacrylonitrile (via an ammoxidation reaction) and/or for conversion of propane to acrylic acid and isobutane to methacrylic acid (via an oxidation reaction). The art known in this field includes numerous patents and patent applications, including for example, U.S. Pat. No. 5,750,760 to Ushikubo et al., U.S. Pat. No. 6,043,185 to Cirjak et al., U.S. Pat. No. 6,156,920 to Brazdil et al., and U.S. Pat. No. 6,514,902 to Inoue et al.

U.S. Pat. No. 6,514,902 describes a catalyst composition containing vanadium, antimony, and small amounts of molybdenum, which requires a special oxidation step during preparation of the catalyst.

Although advancements have been made in the art in connection with catalysts containing molybdenum, vanadium, antimony and other components effective for conversion of propane to acrylonitrile and isobutane to methacrylonitrile (via an ammoxidation reaction) and/or for conversion to acrylic acid and isobutane to methacrylic acid (via an oxidation reaction) the catalysts need further improvement before becoming commercially viable. In general, the art-known catalytic systems for such reactions suffer from generally low yields of the desired product.

Catalysts that produce higher yield of desired product would be desirable. Also desirable would be catalysts that have improved stability under reaction conditions and/or improved resistance to temperature fluctuations in the reactor.

### SUMMARY OF THE INVENTION

The present invention relates to catalyst compositions comprising molybdenum, vanadium, antimony, and at least one other element selected from the group consisting of praesodymium, neodymium, samarium, europium, gadolinium, terbium, dysprosium, holmium, erbium, thulium, ytterbium, and lutetium, and optionally at least one element selected from the group consisting of tungsten, tellurium, titanium, tin, zirconium, and hafnium.

In one embodiment, the invention includes a catalyst composition comprising a mixed oxide of empirical formula

Mo₁VₐSb_{b}X_{c}L_{d}Oₙ

wherein
X is selected from the group consisting of W, Te, Ti, Sn, Zr, Hf, and mixtures thereof,
L is selected from the group consisting of Pr, Nd, Sm, Eu, Gd, Tb, Dy, Ho, Er, Tm, Yb, Lu and mixtures thereof,
0.1≦a≦0.8,
0.01≦b≦0.6,
0≦c≦0.6,
0<d≦0.2,
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, and wherein the catalyst composition contains less than 0.01 moles of niobium relative to one mole of Mo.

In other embodiments X is W, Te, Ti, Sn or mixtures thereof. In other embodiments, X is W.

In other embodiments, L is Nd or Pr.

The present invention also relates to a process for the ammoxidation of a saturated or unsaturated or mixture of saturated and unsaturated hydrocarbon to produce an unsaturated nitrile, the process comprising contacting the saturated or unsaturated or mixture of saturated and unsaturated hydrocarbon with ammonia and an oxygen-containing gas in the presence the catalyst compositions described herein. In one embodiment, the present invention is a process for the ammoxidation of a saturated or unsaturated or mixture of saturated and unsaturated hydrocarbon to produce an unsaturated nitrile, said process comprising contacting the saturated or unsaturated or mixture of saturated and unsaturated hydrocarbon with ammonia and an oxygen-containing gas in the presence of a catalyst composition comprising a mixed oxide of empirical formula:

Mo₁VₐSb_{b}X_{c}L_{d}Oₙ

wherein
X is selected from the group consisting of W, Te, Ti, Sn, Ge, Zr, Hf, and mixtures thereof;
L is selected from the group consisting of La, Pr, Nd, Sm, Eu, Gd, Tb, Dy, Ho, Er, Tm, Yb, Lu and mixtures thereof;
0.1≦a≦0.8,
0.01≦b≦0.6,
0≦c≦0.6,
0<d≦0.2;
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, and wherein the catalyst composition contains less than 0.01 moles of niobium relative to one mole of Mo.

### DETAILED DESCRIPTION OF THE INVENTION

The present invention generally relates to catalyst compositions, methods of preparing such catalyst compositions, and methods of using such catalyst compositions. Such compositions and such catalysts are effective for the ammoxidation of propane to acrylonitrile and isobutane to methacrylonitrile and/or for the oxidation of propane to acrylic acid and isobutane to methacrylic acid.

### Catalyst Composition

In one embodiment, the invention provides a catalyst composition comprising molybdenum, vanadium, antimony, and at least one other element selected from the group consisting of praseodymium, neodymium, samarium, europium, gadolinium, terbium, dysprosium, holmium, erbium, thulium, ytterbium, and lutetium. In certain embodiments, the catalyst composition may include at least one element selected from the group consisting of tungsten, tellurium, titanium, tin, germanium, lanthanum, and halfnium. As used herein, “at least one element selected from the group . . . ” or “at least one lanthanide selected from the group . . . ” includes within its scope mixtures of two or more of the listed elements or lanthanides, respectively.

In one embodiment, the invention is a catalyst composition comprising a mixed oxide of empirical formula:

Mo₁VₐSb_{b}X_{c}L_{d}Oₙ

wherein
X is selected from the group consisting of W, Te, Ti, Sn, Zr, Hf, and mixtures thereof;
L is selected from the group consisting of Pr, Nd, Sm, Eu, Gd, Tb, Dy, Ho, Er, Tm, Yb, Lu and mixtures thereof;
0.1≦a≦0.8,
0.01≦b≦0.6,
0≦c≦0.6,
0<d≦0.2; and
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, and wherein the catalyst composition contains less than 0.01 moles of niobium relative to one mole of Mo.

In one embodiment, the invention is a catalyst composition comprising a mixed oxide of empirical formula:

Mo₁VₐSb_{b}X_{c}L_{d}Oₙ

wherein
X is selected from the group consisting of W, Te, Ti, Sn, and mixtures thereof;
L is selected from the group consisting of Pr, Nd, Sm, Eu, Gd, Tb, Dy, Ho, Er, Tm, Yb, Lu and mixtures thereof;
0.1≦a≦0.8,
0.01≦b≦0.6,
0≦c≦0.6,
0<d ≦0.2; and
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, and wherein the catalyst composition contains less than 0.01 moles of niobium relative to one mole of Mo.

In one or more embodiments, where the catalyst compositions are employed in an ammoxidation process, X may be selected from the group consisting of W, Te, Ti, Ge, Sn, Zr, Hf, and mixtures thereof. In other embodiments, X may be selected from the group consisting of W, Te, Ti, Sn, Zr, Hf, and mixtures thereof. In other embodiments of the catalyst compositions described by the above empirical formulas X is one of W, Te, Ti, or Sn. In other embodiments of the catalyst compositions described by the above empirical formulas X is W.

In one or more embodiments, where the catalyst compositions are employed in an ammoxidation process, L may be selected from the group consisting of La, Pr, Nd, Sm, Eu, Gd, Tb, Dy, Ho, Er, Tm, Yb, and Lu. In other embodiments of the catalyst compositions described by the above empirical formulas, L is Pr, L is Nd, L is Sm, L is Eu, L is Gd, L is Tb, L is Dy, L is Ho, L is Er, L is Tm, L is Yb, and L is Lu. In other embodiments of the catalyst compositions described by the above empirical formulas, L is one of Nd or Pr.

In other embodiments of the catalyst compositions described by the above empirical formulas, the catalyst composition contains less than 0.005 moles of niobium relative to one mole of Mo, and in other embodiments, the catalyst composition contains no niobium. In other embodiments of the catalyst compositions described by the above empirical formulas, the catalyst composition contains less than 0.005 moles of cerium relative to one mole of Mo, and in other embodiments, the catalyst composition contains no cerium. In one or more embodiments of the catalyst compositions described by the above empirical formulas, the catalyst composition contains no tantalum. In one or more embodiments of the catalyst compositions described by the above empirical formulas, the catalyst composition contains no arsenic. In one or more embodiments of the catalyst compositions described by the above empirical formulas, the catalyst composition contains no boron. In one or more embodiments of the catalyst compositions described by the above empirical formulas, the catalyst composition contains no germanium. In one or more embodiments of the catalyst compositions described by the above empirical formulas, the catalyst composition contains no lanthanum.

In other embodiments of the catalyst compositions described by the above empirical formulas, a, b, c, and d are each independently within the following ranges: 0.1<a, 0.2<a, a<0.3, a<0.4, a<0.8, 0.01<b, 0.05<b, 0.1<b, b<0.3, b<0.6, 0≦c, 0.001<c, 0.005<c, c<0.05, c<0.1, c<0.15, c<0.2, c<0.3, c<0.6, 0<d, 0.001 21 d, 0.002<d, 0.003<d, 0.004<d, d<0.006, d<0.01, d<0.02, d<0.05, d<0.1, d<0.2.

In one embodiment of the catalyst compositions described by the above empirical formulas, the catalyst may optionally contain one or more other alkali metals. In this embodiment the catalyst composition comprises a mixed oxide of the empirical formula

Mo₁VₐSb_{b}X_{c}L_{d}AₑOₙ

wherein X, L, a, b, c, d, and n are previously described herein, A is at least one of Li, Na, K, Cs, Rb and mixtures thereof, 0≦e≦0.1, and “e” represents the molar ratio of the corresponding element to one mole of Mo. In another embodiment of the catalyst composition comprising a mixed oxide described by the above empirical formula, the catalyst composition contains no Li, Na, K, Cs, Rb or mixtures thereof (i.e. e equals 0).

The catalyst of the present invention may be made either supported or unsupported (i.e. the catalyst may comprise a support or may be a bulk catalyst). Suitable supports are silica, alumina, zirconia, titania, or mixtures thereof. However, when zirconia or titania are used as support materials then the ratio of molybdenum to zirconium or titanium increases over the values shown in the above formulas, such that the Mo to Zr or Ti ratio is between about 1:11 to 1:10. A support typically serves as a binder for the catalyst resulting in a harder and more attrition resistant catalyst. However, for commercial applications, an appropriate blend of both the active phase (i.e. the complex of catalytic oxides described above) and the support is helpful to obtain an acceptable activity and hardness (attrition resistance) for the catalyst. Directionally, any increase in the amount of the active phase decreases the hardness of the catalyst. The support comprises between 10 and 90 weight percent of the supported catalyst. Typically, the support comprises between 40 and 60 weight percent of the supported catalyst. In one embodiment of this invention, the support may comprise as little as about 10 weight percent of the supported catalyst. In one embodiment of this invention, the support may comprise as little as about 30 weight percent of the supported catalyst. In another embodiment of this invention, the support may comprise as much as about 70 weight percent of the supported catalyst.

### Catalyst Preparation

Advantageously, the synthesis of the catalyst composition described herein can be simplified over preparation methods necessary when the catalyst includes niobium. In one or more embodiments, the catalyst compositions described herein can be prepared by the hydrothermal synthesis methods described herein. Hydrothermal synthesis methods are disclosed in U.S. Patent Application No. 2003/0004379 to Gaffney et al., Watanabe et al., “New Synthesis Route for Mo-V-Nb-Te mixed oxides catalyst for propane ammoxidation”, Applied Catalysis A: General, 194-195, pp. 479-485 (2000), and Ueda et al., “Selective Oxidation of Light Alkanes over hydrothermally synthesized Mo-V-M-O (M=Al, Ga, Bi, Sb and Te) oxide catalysts.”, Applied Catalysis A: General, 200, pp. 135-145, which are incorporated here by reference.

In general, the catalyst compositions described herein can be prepared by hydrothermal synthesis where source compounds (i.e. compounds which contain and/or provide one or more of the metals for the mixed metal oxide catalyst composition) are admixed in an aqueous solution to form a reaction medium and reacting the reaction medium at elevated pressure and elevated temperature in a sealed reaction vessel for a time sufficient to form the mixed metal oxide. In one embodiment, the hydrothermal synthesis continues for a time sufficient to fully react any organic compounds present in the reaction medium, for example, solvents used in the preparation of the catalyst or any organic compounds added with any of the source compounds supplying the mixed metal oxide components of the catalyst composition. This embodiment simplifies further handling and processing of the mixed metal oxide catalyst.

The source compounds are reacted in the sealed reaction vessel at a temperature greater than 100° C. and at a pressure greater than ambient pressure to form a mixed metal oxide precursor. In one embodiment, the source compounds are reacted in the sealed reaction vessel at a temperature of at least about 125° C., in another embodiment at a temperature of at least about 150° C., and in yet another embodiment at a temperature of at least about 175° C. In one embodiment, the source compounds are reacted in the sealed reaction vessel at a pressure of at least about 25 psig, and in another embodiment at a pressure of at least about 50 psig, and in yet another embodiment at a pressure of at least about 100 psig. Such sealed reaction vessels may be equipped with a pressure control device to avoid over pressurizing the vessel and/or to regulate the reaction pressure.

In any case, the source compounds are preferably reacted by a protocol that comprises mixing the source compounds during the reaction step. The particular mixing mechanism is not critical, and can include for example, mixing (e.g., stirring or agitating) the components during the reaction by any effective method. Such methods include, for example, agitating the contents of the reaction vessel, for example by shaking, tumbling or oscillating the component-containing reaction vessel. Such methods also include, for example, stirring by using a stirring member located at least partially within the reaction vessel and a driving force coupled to the stirring member or to the reaction vessel to provide relative motion between the stirring member and the reaction vessel. The stirring member can be a shaft-driven and/or shaft-supported stirring member. The driving force can be directly coupled to the stirring member or can be indirectly coupled to the stirring member (e.g., via magnetic coupling). The mixing is generally preferably sufficient to mix the components to allow for efficient reaction between components of the reaction medium to form a more homogeneous reaction medium (e.g., and resulting in a more homogeneous mixed metal oxide precursor) as compared to an unmixed reaction. This results in more efficient consumption of starting materials and in a more uniform mixed metal oxide product. Mixing the reaction medium during the reaction step also causes the mixed metal oxide product to form in solution rather than on the sides of the reaction vessel. This allows more ready recovery and separation of the mixed metal oxide product by techniques such as centrifugation, decantation, or filtration and avoids the need to recover the majority of product from the sides of the reactor vessel. More advantageously, having the mixed metal oxide form in solution allows for particle growth on all faces of the particle rather than the limited exposed faces when the growth occurs out from the reactor wall.

It is generally desirable to maintain some headspace in the reactor vessel. The amount of headspace may depend on the vessel design or the type of agitation used if the reaction mixture is stirred. Overhead stirred reaction vessels, for example, may take 50% headspace. Typically, the headspace is filled with ambient air which provides some amount of oxygen to the reaction. However, the headspace, as is known the art, may be filled with other gases to provide reactants like O₂ or even an inert atmosphere such as Ar or N₂. The amount of headspace and gas within it depends upon the desired reaction as is known in the art.

The source compounds can be reacted in the sealed reaction vessel at an initial pH of not more than about 4. Over the course of the hydrothermal synthesis, the pH of the reaction mixture may change such that the final pH of the reaction mixture may be higher or lower than the initial pH. In one or more embodiments, the source compounds are reacted in the sealed reaction vessel at a pH of not more than about 3.5. In some embodiments, the components can be reacted in the sealed reaction vessel at a pH of not more than about 3.0, of not more than about 2.5, of not more than about 2.0, of not more than about 1.5 or of not more than about 1.0, of not more than about 0.5 or of not more than about 0. In one or more embodiments, the pH may be from about 0.5 to about 4, in other embodiments, from about 0 to about 4, in yet other embodiments, from about 0.5 to about 3.5. In some embodiments, the pH is from about 0.7 to about 3.3, and in certain embodiments, from about 1 to about 3. The pH may be adjusted by adding acid or base to the reaction mixture.

The source compounds can be reacted in the sealed reaction vessels at the aforementioned reaction conditions (including for example, reaction temperatures, reaction pressures, pH, stirring, etc., as described above) for a period of time sufficient to form the mixed metal oxide, preferably where the mixed metal oxide comprises a solid state solution comprising the required elements as discussed above, and at least a portion thereof preferably having the requisite crystalline structure for active and selective propane or isobutane oxidation and/or ammoxidation catalysts, as described below. The exact period of time is not narrowly critical, and can include for example at least about three hours, at least about six hours, at least about twelve hours, at least about eighteen hours, at least about twenty-four hours, at least about thirty hours, at least about thirty-six hours, at least about forty-two hours, at least about forty-eight hours, at least about fifty-four hours, at least about sixty hours, at least about sixty-six hours or at least about seventy-two hours. Reaction periods of time can be even more than three days, including for example at least about four days, at least about five days, at least about six days, at least about seven days, at least about two weeks or at least about three weeks or at least about one month.

Following the reaction step, further steps of the catalyst preparation methods may include work-up steps, including for example cooling the reaction medium comprising the mixed metal oxide (e.g., to about ambient temperature), separating the solid particulates comprising the mixed metal oxide from the liquid (e.g., by centrifuging and/or decanting the supernatant, or alternatively, by filtering), washing the separated solid particulates (e.g., using distilled water or deionized water), repeating the separating step and washing steps one or more times, and effecting a final separating step. In one embodiment, the work up step comprises drying the reaction medium, such as by rotary evaporation, spray drying, freeze drying etc. This eliminates the formation of a metal containing waste stream.

After the work-up steps, the washed and separated mixed metal oxide can be dried. Drying the mixed metal oxide can be effected under ambient conditions (e.g., at a temperature of about 25° C. at atmospheric pressure), and/or in an oven, for example, at a temperature ranging from about 40° C. to about 150° C., and preferably of about 120° C. over a drying period of about time ranging from about five to about fifteen hours, and preferably of about twelve hours. Drying can be effected under a controlled or uncontrolled atmosphere, and the drying atmosphere can be an inert gas, an oxidative gas, a reducing gas or air, and is typically and preferably air.

As a further preparation step, the dried mixed metal oxide can be treated to form the mixed metal oxide catalyst. Such treatments can include for example calcinations (e.g., including heat treatments under oxidizing or reducing conditions) effected under various treatment atmospheres. The work-up mixed metal oxide can be crushed or ground prior to such treatment, and/or intermittently during such pretreatment. In one embodiment, for example, the dried mixed metal oxide can be optionally crushed, and then calcined to form the mixed metal oxide catalyst. The calcination may be effected in an inert, reducing, or oxidizing atmosphere. In one embodiment, the calcination is effected in an inert atmosphere such as nitrogen. In one or more embodiments, the calcination conditions include temperatures ranging from about 400° C. to about 700° C., in other embodiments, from about 500° C. to about 650° C., and in some embodiments, the calcination temperature may be about 600° C.

The treated (e.g., calcined) mixed metal oxide can be further mechanically treated, including for example by grinding, sieving and pressing the mixed metal oxide into its final form for use in fixed bed or fluid bed reactors. In other embodiments, the catalyst may be shaped into its final form prior to any calcinations or other heat treatment. For example, in the preparation of a fixed bed catalyst, the catalyst precursor slurry is typically dried by heating at an elevated temperature and then shaped (e.g. extruded, pellitized, etc.) to the desired fixed bed catalyst size and configuration prior to calcination. Similarly, in the preparation of fluid bed catalysts, the catalyst precursor slurry may be spray dried to yield microspheroidal catalyst particles having particle diameters in the range from 10 to 200 microns and then calcined.

Some source compounds containing and providing the metal components used in the synthesis of the catalyst (also referred to herein as a “source” or “sources”) can be provided to the reaction vessel as aqueous solutions of the metal salts. Some source compounds of the metal components can be provided to the reaction vessels as solids or as slurries comprising solid particulates dispersed in an aqueous media. Some source compounds of the metal components can be provided to the reaction vessels as solids or as slurries comprising solid particulates dispersed in non-aqueous solvents or other non-aqueous media.

Suitable source compounds for synthesis of the catalysts as described herein include the following. A suitable molybdenum source may include molybdenum (VI) oxide (MoO₃), ammonium heptamolybdate or molybdic acid. A suitable vanadium source may include vanadyl sulfate, ammonium metavanadate, or vanadium (V) oxide. A suitable antimony source may include antimony (III) oxide, antimony (III) acetate, antimony (III) oxalate, antimony (V) oxide, antimony (III) sulfate, or antimony (III) tartrate.

A suitable tungsten source may include ammonium metatungstate, tungstic acid, and tungsten trioxide. A suitable tellurium source may include telluric acid, tellurium dioxide, tellurium trioxide, organic tellurium compounds such as methyltellurol and dimethyl tellurol.

A suitable titanium source may include rutile and/or anatase titanium dioxide (TiO₂), e.g. Degussa P-25, titanium isopropoxide, TiO(oxalate), TiO(acetylacetonate)₂, or titanium alkoxide complexes, such as Tyzor 131. A suitable tin source may include tin (II) acetate. A suitable germanium source may include germanium (IV) oxide. A suitable zirconium source may include zirconyl nitrate or zirconium (IV) oxide. A suitable hafnium sources may include hafnium (IV) chloride or hafnium (IV) oxide.

Suitable praseodymium sources may include praseodymium (III) chloride, praseodymium (III, IV) oxide, praseodymium (III) isopropoxide, and praseodymium (III) acetate hydrate. Suitable neodymium sources may include neodymium (III) chloride, neodymium (III) oxide, neodymium (III) isopropoxide, and neodymium (III) acetate hydrate. Suitable samarium sources may include samarium (III) chloride, samarium (III) oxide, samarium (III) isopropoxide, and samarium (III) acetate hydrate. Suitable europium sources may include europium (III) chloride, europium (III) oxide, and europium (III) acetate hydrate. Suitable gadolinium sources may include gadolinium (III) chloride, gadolinium (III) oxide, and gadolinium (III) acetate hydrate. Suitable terbium sources include terbium (III) chloride, terbium (III) oxide, and terbium (III) acetate hydrate. Suitable dysprosium sources may include dysprosium (III) chloride, dysprosium (III) oxide, dysprosium (III) isopropoxide, and dysprosium (III) acetate hydrate. Suitable holmium sources may include holmium (III) chloride, holmium (III) oxide, and holmium (III) acetate hydrate. Suitable erbium sources may include erbium (III) chloride, erbium (III) oxide, erbium (III) isopropoxide, and erbium (III) acetate hydrate. Suitable thulium sources may include thulium (III) chloride, thulium (III) oxide, and thulium (III) acetate hydrate. Suitable ytterbium sources may include ytterbium (III) chloride, ytterbium (III) oxide, ytterbium (III) isopropoxide, and ytterbium (III) acetate hydrate. Suitable sources of lutetium may include lutetium (III) chloride, lutetium (III) oxide, and lutetium (III) acetate hydrate. Nitrates of the above listed metals may also be employed as source compounds.

The amount of aqueous solvent in the reaction medium may vary due to the solubilities of the source compounds combined to form the particular mixed metal oxide. The amount of aqueous solvent should at least be sufficient to yield a slurry (a mixture of solids and liquids which is able to be stirred) of the reactants. It is typical in hydrothermal synthesis of mixed metal oxides to leave an amount of headspace in the reactor vessel.

Variations on the above methods will be recognized by those skilled in the art. For example a method for preparing the catalyst described herein having the following empirical formula:

Mo V_{0.1-0.3}Sb_{0.1-0.3} W_{0.01-0.05}Nd_{0.005-0.02}Oₙ

in which “n” is determined by the oxidized states of the other elements, comprises preparing solutions or slurries of source compounds for the catalyst. In one or the first slurry, molybdenum trioxide (MoO₃), vanadium pentoxide (V₂O₅), antimony oxide (Sb₂O₃), ammonium tungstate (NH₄)₆W₁₂O₃₉ and neodymium acetate hydrate (Nd(O₂CCH₃)₃×H₂O) are dissolved/slurried in water at the desired ratios (all ratios are relative to molybdenum metal). Oxalic acid (HO₂CCO₂H) is added. The volume of the solution/slurry is adjusted with water. The solution/slurry is heated with mixing to 175° C. and held at this temperature for 48 hours, and then cooled to room temperature, typically by natural heat dissipation. The cooled slurry is filtered to remove the mother liquor, and the remaining solids are washed and then dried and then calcined under nitrogen at 600° C. to activate the catalyst. The calcined catalyst is pulverized, then pelletized and sized, or spray dried for testing and/or ultimate use.

### Conversion of Propane and Isobutane via Ammoxidation and Oxidation Reaction

Propane is preferably converted to acrylonitrile and isobutane to methacrylonitrile, by providing one or more of the aforementioned catalysts in a gas-phase flow reactor, and contacting the catalyst with propane or isobutane in the presence of oxygen (e.g. provided to the reaction zone in a feedstream comprising an oxygen-containing gas, such as and typically air) and ammonia under reaction conditions effective to form acrylonitrile or methacrylonitrile. For this reaction, the feed stream preferably comprises propane or isobutane, an oxygen-containing gas such as air, and ammonia. In one or more embodiments, the molar ratio of propane or isobutane to oxygen is from about 0.125 to about 5, in another embodiment, from about 0.25 to about 4.5, and in another embodiment, from about 0.35 to about 4. In one or more embodiments, the molar ratio of propane or isobutane to ammonia is from about 0.3 to about 4, and in another embodiment, from about 0.5 to about 3. The feed stream can also comprise one or more additional feed components, including acrylonitrile or methacrylonitrile product (e.g., from a recycle stream or from an earlier-stage of a multi-stage reactor), and/or steam. For example, the feedstream can comprise about 5% to about 30% by weight relative to the total amount of the feed stream, or by mole relative to the amount of propane or isobutane in the feed stream. In one embodiment the catalyst compositions described herein are employed in the ammoxidation of propane to acrylonitrile is a once-through process, i.e., it operates without recycle of recovered but unreacted feed materials.

Propane can also be converted to acrylic acid and isobutane to methacrylic acid by providing one or more of the aforementioned catalysts in a gas-phase flow reactor, and contacting the catalyst with propane in the presence of oxygen (e.g. provided to the reaction zone in a feedstream comprising an oxygen-containing gas, such as and typically air) under reaction conditions effective to form acrylic acid. The feed stream for this reaction preferably comprises propane or isobutane to oxygen ranging from about 0.15 to about 5, and preferably from about 0.25 to about 2. The feed stream can also comprise one or more additional feed components, including acrylic acid or methacrylic acid product (e.g. from a recycle stream or from an earlier-stage of a multi-stage reactor), and/or steam. For example, the feedsteam can comprise about 5% to about 30% by weight relative to the total amount of the feed stream, or by mole relative to the amount of propane isobutane in the feed stream.

The specific design of the gas-phase flow reactor is not narrowly critical. Hence, the gas-phase flow reactor can be a fixed-bed reactor, a fluidized-bed reactor, or another type of reactor. The reactor can be a single reactor, or can be one reactor in a multi-stage reactor system. Preferably, the reactor comprises one or more feed inlets for feeding a reactant feedstream to a reaction zone of the reactor, a reaction zone comprising the mixed metal oxide catalyst, and an outlet for discharging reaction products and unreacted reactants.

The reaction conditions are controlled to be effective for converting the propane to acrylonitrile or acrylic acid or for converting the isobutane to methacrylonitrile or methacrylic acid respectively, or the isobutane to methacrylonitrile. Generally, reaction conditions include a temperature ranging from about 300° C. to about 550° C., preferably from about 325° C. to about 500° C., and in some embodiments from about 350° C. to about 450° C., and in other embodiments from about 430° C. to about 520° C. The pressure of the reaction zone can be controlled to range from about 0 psig to about 200 psig, preferably from about 0 psig to about 100 psig, and in some embodiments from about 0 psig to about 50 psig.

Generally, the flow rate of the propane or isobutene containing feedstream through the reaction zone of the gas-phase flow reactor can be controlled to provide a weight hourly space velocity (WHSV) ranging from about 0.02 to about 5, in some embodiments from about 0.05 to about 1, and in other embodiments from about 0.1 to about 0.5, in each case, for example, in grams propane or isobutane to grams of catalyst per hour.

The resulting acrylonitrile and/or acrylic acid or methacrylonitrile and/or methacrylic acid product can be isolated, if desired, from other side-products and/or from unreacted reactants according to method known in the art.

The catalyst compositions described herein when employed in the single pass (i.e. no recycle) ammoxidation of propane are capable of producing a yield of about 45-52 percent acrylonitrile, or higher, with a selectivity of about 20% to COₓ (carbon dioxide+carbon monoxide), and a selectivity of about 15% to a mixture of hydrogen cyanide (HCN) and acetonitrile or methyl cyanide (CH₃CN). The effluent of the reactor may also include unreacted oxygen (O₂), ammonia (NH₃), nitrogen (N₂), helium (He), and entrained catalyst fines.

Advantageously, in one or more embodiments, the yield of unsaturated nitrile does not decrease after the catalyst is exposed to fluctuations in reactor temperature, including reactor temperatures of greater than 440° C.

### SPECIFIC EMBODIMENTS

In order to illustrate the instant invention, samples of a base catalyst, with and without various catalyst modifiers, were prepared and then evaluated under similar reaction conditions. The compositions listed below are nominal compositions, based on the total metals added in the catalyst preparation. Since some metals may be lost or may not completely react during the catalyst preparation, the actual composition of the finished catalyst may vary slightly from the nominal compositions shown below.

### Comparative Example #1—MO₁V_{0.25}Sb_{0.2}Oₙ

A 125 mL Teflon reactor liner was loaded with MoO₃ (8.75 g), V₂O₅ (1.38 g), Sb₂O₃ (1.772 g), and water (20 mL). The mixture was stirred and 1.0 M oxalic acid (24.3 mL) was added. Water was added to obtain an about 75% fill volume in the reactor liner. The reactor was then sealed with a Teflon cap in a metal housing, placed in an oven preheated to 175° C. and continuously rotated to effect mixing of the liquid and solid reagents. After 48 h (h=hours), the reactor was cooled and the Teflon liner was removed from the housing. The product mixture was centrifuged and the supernatant was decanted to waste. The remaining solids were washed by the addition of water in two portions. The wet solid was then dried in air at 120° C. for 12 h. The resulting solid material was crushed and calcined under N₂ for 2 h at 600° C. The solid was then ground, pressed, and sieved to a particle size range of 145 to 355 microns and tested for catalytic performance. This material has the nominal composition Mo₁V_{0.25}Sb_{0.2}Oₙ.

The material was tested as a catalyst for the heterogeneous ammoxidation of propane to acrylonitrile. At 430° C., WHSV=0.15 and a feed ratio of C₃H₈/NH₃/O₂/He=1/1.2/3/12, an acrylonitrile yield of 45% was obtained (77% propane conversion, 58% acrylonitrile selectivity).

### Example #2—Mo₁V_{0.28}Sb_{0.17}W_{0.02}Nd_{0.01}Oₙ

A 23 mL Teflon reactor liner was loaded with MoO₃ (1.151 g), V₂O₅ (0.204 g), Sb₂O₃ (0.198 g), (NH₄)₆W₁₂O₃₉ (0.039 g), Nd(OAc)₃ (0.026 g), and water (5 mL). As used in this example and several subsequent examples, “(OAc)₃” designates the acetate hydrate for the named lanthanide metal. The mixture was stirred and 0.5 M oxalic acid (6.4 mL) was added. Water was added to obtain an about 75% fill volume in the reactor liner. The reactor was then sealed with a Teflon cap in a metal housing, placed in an oven preheated to 175° C. and continuously rotated to effect mixing of the liquid and solid reagents. After 48 h, the reactor was cooled and the Teflon liner was removed from the housing. The product mixture was centrifuged and the supernatant was decanted to waste. The remaining solids were washed by the addition of water in two portions. The wet solid was then dried in air at 120° C. for 12 h. The resulting solid material was crushed and calcined under N₂ for 2 h at 600° C. The solid was then ground, pressed, and sieved to a particle size range of 145 to 355 microns and tested for catalytic performance. This material has the nominal composition Mo₁V_{0.28}Sb_{0.17}W_{0.02}Nd_{0.01}Oₙ.

The material was tested as a catalyst for the heterogeneous ammoxidation of propane to acrylonitrile. At 430° C., WHSV=0.25 (higher than the WHSV of 0.1 at which Comparative Example #1 was tested) and a feed ratio of C₃H₈/NH₃/O₂/He=1/1.2/3/12, an acrylonitrile yield of 45% was obtained (86% propane conversion, 52% acrylonitrile selectivity).

### Example #3—Mo₁V_{0.28}Sb_{0.23}Wo_{0.02}Nd_{0.01}Oₙ

A 125 mL Teflon reactor liner was loaded with MoO₃ (8.00 g), V₂O₅ (1.415 g), Sb₂O₃ (1.863 g), (NH₄)₆W₁₂O₃₉ (0.272 g), Nd(OAc)₃ (0.179 g), and water (10 mL). The mixture was stirred and 0.5 M oxalic acid (44.5 mL) was added. Water was added to obtain an about 80% fill volume in the reactor liner. The reactor was then sealed with a Teflon cap in a metal housing, placed in an oven preheated to 175° C. and continuously rotated to effect mixing of the liquid and solid reagents. After 48 h, the reactor was cooled and the Teflon liner was removed from the housing. The product mixture was centrifuged and the supernatant was decanted to waste. The remaining solids were washed by the addition of water in two portions. The wet solid was then dried in air at 120° C. for 12 h. The resulting solid material was crushed and calcined under N₂ for 2 h at 600° C. The solid was then ground, pressed, and sieved to a particle size range of 145 to 355 microns and tested for catalytic performance. This material has the nominal composition Mo₁V_{0.28}Sb_{0.23}W_{0.02}Nd_{0.01}Oₙ.

The material was tested as a catalyst for the heterogeneous ammoxidation of propane to acrylonitrile. At 460° C., WHSV=0.2 and a feed ratio of C₃H₈/NH₃/O₂/He=1/2.4/3/12, an acrylonitrile yield of 51% was obtained (78% propane conversion, 66% acrylonitrile selectivity).

### Example #4—Mo₁V_{0.28}Sb_{0.23}W_{0.02}Nd_{0.005}Oₙ

A 23 mL Teflon reactor liner was loaded with MoO₃ (1.0 g), V₂O₅ (0.177 g), Sb₂O₃ (0.233 g), ammonium metatungstate (0.034 g), Nd(OAc)₃ (0.024 g), oxalic acid (0.35 g) and water (8.2 mL). Water was added to obtain an about 80% fill volume in the reactor liner. The reactor was then sealed with a Teflon cap in a metal housing, placed in an oven preheated to 175° C. and continuously rotated to effect mixing of the liquid and solid reagents. After 48 h, the reactor was cooled to room temperature, and the Teflon liner was removed from the housing. The slurry was transferred to a round bottom flask. The liquid was removed using a rotary evaporator. The solid was further dried in air at 120° C. for 12 h. The solid material was then crushed and calcined under N₂ for 2 h at 600° C. The solid was then ground, pressed, and sieved to a particle size range of 145 to 355 microns and tested for catalytic performance. This material has the nominal composition Mo₁V_{0.28}Sb_{0.23}W_{0.02}Nd_{0.005}Oₙ.

The material was tested as a catalyst for the heterogeneous ammoxidation of propane to acrylonitrile. As shown in Table I, the catalyst was tested at temperatures of between 445 and 453° C., WHSV=0.2 and a feed ratio of C₃H₈/NH₃/O₂/N₂=1/2.2/3.15/12. Acrylonitrile yields of 49 to 51% were obtained. Additional testing of the sample as a heterogeneous propane ammoxidation catalyst was then performed at temperatures as high as 470° C., after which the temperature was lowered and the catalyst was tested at temperatures from 446 to 452° C., WHSV=0.2 and a feed ratio of C₃H₈/NH₃/O₂/N₂=1/2.2/3.15/12. Acrylonitrile yields of 49 to 51% were again obtained, as shown in Table I.

At 459° C., WHSV=0.2 and a feed ratio of C₃H₈/NH₃/O₂/N₂=1/2.4/3.15/12, an acrylonitrile yield of 52% was obtained (78% propane conversion, 66% acrylonitrile selectivity).

### Comparative Example #5—Mo₁V_{0.3}Sb_{0.2}Nb_{0.06}Ti_{0.1}Ce_{0.005}Oₙ

Into a 50 mL beaker that contained a magnetic stir bar was placed VOSO₄.3H₂O (0.977 g) and 6 mL water. After the vanadium reagent dissolved, Sb₂O₃ (0.437 g) was added and the mixture was stirred for 15 minutes. To the resulting slurry was added MoO₃ (2.157 g), TiO₂ (0.120 g), Ce(OAc)₃.1.5H₂O (0.026 g), and niobium oxalate (2.250 mL of a 0.40 M solution, where the molar ratio of oxalate to niobium is about 3/1). The contents of the beaker (except the stir bar) were transferred to a 23 mL Teflon reactor liner, using 0.8 mL of additional water to effect complete transfer. The reactor liner was sealed with a Teflon cap in a metal housing, placed in an oven preheated to 175° C. and continuously rotated to effect mixing of the liquid and solid reagents. After 46 h the reactor was cooled and the Teflon liner was removed from the housing. The product slurry was filtered to separate the solid reaction products from the reaction slurry. The filtrate was washed with two 50 mL portions of 60° C. distilled water followed by filtration. The wet solid was dried in air at 90° C. for 12 h. The resulting solid material was crushed and calcined under N₂ for 2 h at 600° C. The solid was then ground, pressed, and sieved to a particle size range of 145 to 355 microns and tested for catalytic performance. The nominal composition of the material is Mo₁V_{0.3}Sb_{0.2}Nb_{0.06}Ti_{0.1}Ce_{0.005}Oₙ.

The material was tested as a catalyst for the heterogeneous ammoxidation of propane to acrylonitrile by using exactly the same procedure and conditions as for Example 4. As shown in Table I, between 445 and 453° C., WHSV=0.2 and a feed ratio of C₃H₈/NH₃/O₂/N₂=1/2.2/3.15/12, acrylonitrile yields of 49 to 55% were obtained. Additional testing of the sample as a heterogeneous propane ammoxidation catalyst was then performed at temperatures as high as 470° C., after which the temperature was lowered and the catalyst was tested at temperatures of from 446 to 452° C., WHSV=0.2 and a feed ratio of C₃H₈/NH₃/O₂/N₂=1/2.2/3.15/12. Acrylonitrile yields were only 47 to 48% following exposure of the sample to the high temperature ammoxidation conditions, as shown in Table I.

TABLE I

| Initial | | Performance After | |
|---|---|---|---|
| Performance | | High Temperature | |
| Ammoxidation | | Ammoxidation | |
| Temperature ° C. | AN Yield % | Temperature ° C. | AN Yield % |

Example #4 -- Mo_{1.0}V_{0.28}Sb_{0.23}W_{0.02}Nd_{0.01}Oₙ

| | | | |
|---|---|---|---|
| 445 | 49 | 446 | 49 |
| 449 | 50 | 447 | 50 |
| 451 | 51 | 450 | 51 |
| 453 | 51 | 452 | 51 |

Comparative Example #5 -- Mo_{1.0}V_{0.3}Sb_{0.2}Nb_{0.06}Ti_{0.1}Ce_{0.005}Oₙ

| | | | |
|---|---|---|---|
| 445 | 55 | 446 | 48 |
| 449 | 52 | 448 | 48 |
| 451 | 50 | 450 | 48 |
| 453 | 49 | 452 | 47 |

While the foregoing description and the above embodiments are typical for the practice of the instant invention, it is evident that many alternatives, modifications, and variations will be apparent to those skilled in the art in light of this description. Accordingly, it is intended that all such alternatives, modifications and variations are embraced by and fall within the spirit and broad scope of the appended claims.

We claim:

1. A catalyst composition comprising a mixed oxide of empirical formula:
Mo₁VₐSb_{b}X_{c}L_{d}Oₙ
wherein X is selected from the group consisting of W, Te, Ti, Sn, Zr, Hf, and mixtures thereof; L is selected from the group consisting of Pr, Nd, Sm, Eu, Gd, Tb, Dy, Ho, Er, Tm, Yb, Lu and mixtures thereof;
0.1≦a≦0.8,
0.01≦b≦0.6,
0≦c≦0.6,
0<d≦0.2;
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, and wherein the catalyst composition contains less than 0.01 moles of niobium relative to one mole of Mo.

2. The catalyst composition of claim 1, wherein 0.1<a<0.8, 0.01<b<0.6, 0.001<c<0.3, and 0.001<d<0.1.

3. The catalyst composition of claim 1, wherein X is selected from the group consisting of elements W, Te, Ti, Sn and mixtures thereof.

4. The catalyst composition of claim 1, wherein X is W.

5. The catalyst composition of claim 1, wherein L is selected from the group consisting of elements Nd, Pr and mixtures thereof.

6. The catalyst composition of claim 1, wherein the catalyst composition comprises a support selected from the group consisting of silica, alumina, zirconia, titania, or mixtures thereof.

7. The catalyst composition of claim 6, wherein the support comprises about 10 to about 70 weight percent of the catalyst.

8. The catalyst composition of claim 1, wherein the composition further comprises one or more alkali elements, and may be represented by empirical formula:
Mo₁VₐSb_{b}X_{c}L_{d}AₑOₙ
wherein A is Li, Na, K, Cs, Rb or a mixture thereof, 0≦e≦0.1, and e represents the molar ratio of the corresponding element to one mole of Mo.

9. A catalyst composition comprising a mixed oxide of empirical formula:
Mo₁VₐSb_{b}X_{c}L_{d}Oₙ
wherein
X is W; L is selected from the group consisting of Nd, Pr, and mixtures thereof,
0.1≦a≦0.8,
0.01≦b≦0.6,
0≦c≦0.6,
0<d≦0.04;
n is the number of oxygen atoms required to satisfy valance requirements of all other elements present in the mixed oxide with the proviso that one or more of the other elements in the mixed oxide can be present in an oxidation state lower than its highest oxidation state, and a, b, c, and d represent the molar ratio of the corresponding element to one mole of Mo, and wherein the catalyst composition contains less than 0.01 moles of niobium relative to one mole of Mo.

10. The catalyst composition of claim 9, wherein 0.1<a<0.8, 0.01<b<0.6, 0.001<c<0.3, and 0.001<d<0.1.

11. The catalyst composition of claim 9, wherein the composition further comprises one or more alkali elements, and may be represented by empirical formula:
Mo₁VₐSb_{b}X_{c}L_{d}AₑOₙ
wherein A is Li, Na, K, Cs, Rb or a mixture thereof, 0≦e≦0.1, and e represents the molar ratio of the corresponding element to one mole of Mo.

12. The catalyst composition of claim 9, wherein the catalyst composition comprises a support selected from the group consisting of silica, alumina, zirconia, titania, or mixtures thereof.

13. The catalyst composition of claim 12, wherein the support comprises about 10 to about 70 weight percent of the catalyst.

14. A process for the ammoxidation of a saturated or unsaturated or mixture of saturated and unsaturated hydrocarbon to produce an unsaturated nitrile, said process comprising contacting the saturated or unsaturated or mixture of saturated and unsaturated hydrocarbon with ammonia and an oxygen-containing gas in the presence of a catalyst composition comprising a mixed oxide of empirical formula:
Mo₁VₐSb_{b}X_{c}L_{d}Oₙ
wherein
X is selected from the group consisting of W, Te, Ti, Sn, Ge, Zr, Hf, and mixtures thereof; L is selected from the group consisting of La, Pr, Nd, Sm, Eu, Gd, Tb, Dy, Ho, Er, Tm, Yb, Lu and mixtures thereof;
0.1≦a≦0.8,
0.01≦b≦0.6,
0≦c≦0.6,
0<d≦0.2;
n is the number of oxygen atoms required to satisfy valance requirements of all other elements present in the mixed oxide with the proviso that one or more of the other elements in the mixed oxide can be present in an oxidation state lower than its highest oxidation state, a, b, c, and d represent the molar ratio of the corresponding element to one mole of Mo, wherein the catalyst composition contains less than 0.01 moles of niobium relative to one mole of Mo, and wherein the catalyst composition contains less than 0.01 moles of cerium relative to one mole of Mo.

15. The process of claim 14, wherein in the catalyst composition: 0.1<a<0.8, 0.01<b<0.6, 0.001<c<0.3, and 0.001<d<0.1.

16. The process of claim 14, wherein X is selected from the group consisting of elements W, Te, Ti, Sn and mixtures thereof.

17. The process of claim 14, wherein X is W.

18. The process of claim 14, wherein L is selected from the group consisting of elements Nd, Pr and mixtures thereof.

19. The process of claim 14, wherein the catalyst composition comprises a support selected from the group consisting of silica, alumina, zirconia, titania, or mixtures thereof.

20. The process of claim 19, wherein the support comprises about 10 to about 70 weight percent of the catalyst.
